(19) United States
(12) Patent Application Publication
(10) Pub. No.: US 2017/0269067 A1
(43) Pub. Date: Sep. 21, 2017

(54) USE OF FUSION PROTEINS TO IMPROVE THE AVAILABILITY OF ANTIGENIC PEPTIDE EPITOPES IN IMMUNOASSAYS

(71) Applicants: Charles L. GINSBURGH, San Mateo, CA (US); Sricharan BANDHAKAVI, South San Francisco, CA (US); Youngchool CHOE, Millbrae, CA (US); Yan LIU, Foster City, CA (US)

(72) Inventors: Charles L. GINSBURGH, San Mateo, CA (US); Sricharan BANDHAKAVI, South San Francisco, CA (US); Youngchool CHOE, Millbrae, CA (US); Yan LIU, Foster City, CA (US)

(21) Appl. No.: 15/462,498

(22) Filed: Mar. 17, 2017

Related U.S. Application Data
(60) Provisional application No. 62/309,909, filed on Mar. 17, 2016.

(51) Int. Cl. G01N 33/53

(52) U.S. Cl. CPC G01N 33/5306; G01N 2333/4709; G01N 2333/58

(57) ABSTRACT

Methods, techniques, systems and compositions to reduce or prevent the aggregation and/or epitope masking of control/calibration peptides. The methods described herein may be particularly helpful for calibration and quantifying target proteins from immunoassays such as solid-phase enzyme immunoassays (EIAs).

### CROSS REFERENCE TO RELATED APPLICATIONS

This patent application claims priority to U.S. Provisional Patent Application No. 62/309,909, filed on Mar. 17, 2016 and titled “USE OF FUSION PROTEINS TO IMPROVE THE AVAILABILITY OF ANTIGENIC PEPTIDE EPITOPES IN IMMUNOASSAYS.” This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Mar. 17, 2017, is named 14381-713_201_SL.TXT and is 3,859 bytes in size.

### FIELD

The methods and compositions described herein address the use of fusion proteins to improve the availability of antigenic peptide epitopes in immunoassays.

### BACKGROUND

In many scientific and/or medical applications (such as diagnostic assays) the identification and prevalence of biologically relevant peptides in serum, plasma or other bodily fluids yields critical insight into potential or current disease states of the sample donor. In immunoassay assay formats, the use of synthetically derived peptides as calibrators and/or as controls allows the analytical quantitation of these peptide analytes. In many cases the peptide aggregates or assembles into forms which mask sequence-specific antigenic epitopes. In these cases, the peptide aggregate becomes a poor or unusable calibrant/control.

Although it is known to use biochemical agents to aid in the disaggregation of proteins in peptide diluents to improve the solubility (e.g., disaggregation) of peptides when used as calibrators and/or controls, the effectiveness of such techniques is often inconsistent or requires a solution composition that may interfere with the chemistries of other parts of the assay (e.g., of an EIA assay). Further, such techniques may not be fully effective in disaggregating the peptide.

Thus, it would be useful to provide methods, techniques, systems and compositions that are able to reduce or prevent the aggregation and/or epitope masking of control/calibration peptides. Describes herein are techniques (e.g., methods) and systems (e.g., kits, compositions, and the like) that may address these problems.

### SUMMARY OF THE DISCLOSURE

Described herein are methods whereby specific target peptides are fused to soluble proteins thereby reducing the aggregation state of the target peptides. This will subsequently unmask critical specific antigenic epitopes, improving the utility of the peptide to act as a calibrant. In addition, these fused peptides are more susceptible to the solubilization action of several classical biochemical agents such as salts, detergents and chaotropic agents.

This techniques and systems described herein can be applied to any peptide or protein material in which the aggregation of the peptide limits the applicability and/or efficiency of the peptide's use as a calibrant or control.

In the description below, the highly soluble (e.g., solubilizing) protein to which the synthetic target protein (or a synthetic immunogenic region or portion of a target protein) is typically Glutathione S-transferase (GST), however any other protein region (typically between 10 and 500 amino acids long, e.g., between 10 and 200 aa, between 10 and 100 aa, between 10 and 40 aa, etc.) may be used. The soluble protein typically has a solubility in salt and/or detergent that is approximately the same or greater than GST, such as GBI (protein G B1 domain, 56 residues), protein D (110 residues), the Z domain of Staphylococcal protein A (58 residues) and thioredoxin (109 residues). While the use of such solubility-enhancing proteins or soluble portions of such proteins as fusions to increase the solubility of synthetic proteins is known, the use of such proteins to increase the epitope exposure so that they may be used as a calibrant in the same concentrations as natural native (e.g., extracted) target protein to provide more accurate estimates of target protein concentration is surprising.

As used herein “synthetic” proteins are proteins that are formed by recombinant techniques, including bacterial, yeast or other cell culture techniques and may include proteins that have been modified (e.g., to increase expression), including by fusion to the soluble proteins as described herein. In contrast, native or naturally isolated proteins refer to proteins isolated from a human/animal or a primary tissue culture from a human/animal.

For example, described herein are methods of detecting a target protein from a patient sample using a synthetic calibration protein having increased solubility. Such methods may include: performing an immunoassay on the patient sample using an antibody to the target protein; generating a plurality of calibration solutions comprising different concentrations of a fusion protein of a synthetic target protein and Glutathione S-transferase (GST) in a detergent solution; and using the plurality of calibration solution to calibrate the immunoassay and provide a concentration of the target protein, wherein the different concentrations of the calibration solutions correspond to molar concentrations of the fusion protein without value-assigning the concentrations to determine the concentration of the target protein.

Any of these methods may also include using the plurality of calibration solutions to make a calibration curve. The step of providing a concentration of the target protein (e.g., from the immunoassay) may therefore include using a calibration curve based on known concentrations of the synthetic fusion proteins including the solubilizing protein (e.g., GST).

In general, the immunoassays described herein may be any quantifiable immunoassay, including, but not limited to solid-phase enzyme immunoassay (EIA).

A patient sample may be any tissue or fluid sample (e.g., blood, saliva, mucus, etc.). For example, the patient sample may be a blood sample.

The target protein may be any appropriate target protein, particularly those for which, when synthesized by any method (e.g., bacterial expression, yeast expression, etc.) aggregate and/or mask or otherwise have a lower epitope exposure than native/naturally isolated target protein. Example of target proteins include, but are not limited to: NT-proBNP, Beta Amyloid peptide (AB42), MR-proANP, etc.

The fusion proteins described herein may include other tagged or functional regions. For example any of these proteins may include a His6 Tag (SEQ ID NO: 3), a TEV cleavage site (e.g., between the target protein and the GST), etc. Alternatively the fusion protein may include only the soluble protein/protein portion and the target protein or portion of the target protein.

The fusion protein may have the soluble protein (e.g., GST) on either the C-eterminal or the N-terminal. In some variations it may be particularly helpful to have the GST fused to the N-terminal portion of the target protein.

The detergent solution may include a salt and/or other solubilizing agent to help solubilize the fusion protein. For example, the detergent solution may include one or more of: an anionic surfactant, a nonionic surfactant, a zwitterionic detergent, and a chaotropic agent. An anionic surfactant may include (Sodium dodecyl sulfate (SDS)), a nonionic surfactant may include Triton X-100, Tergitol-type NP-40 (NP-40), and/or octylphenoxypolyethoxyethanol (IGEPAL CA-630); a zwitterionic detergent may include 3-[(3-Cholamidopropyl)dimethylammonio]-1-propanesulfonate (CHAPS); and a chaotropic agent may be Guanidine Hydrochloride.

As mentioned, the methods described herein may be particularly helpful for calibrating and detecting NT-proBNP. For example, a method of detecting NT-proBNP from a patient sample using a synthetic calibration NT-proBNP having increased solubility may include: performing an immunoassay on the patient sample using an antibody to NT-proBNP; generating a plurality of calibration solutions comprising different concentrations of a fusion protein of a synthetic NT-proBNP and Glutathione S-transferase (GST) in a detergent solution; and using the plurality of calibration solutions to calibrate the immunoassay and provide a concentration of the NT-proBNP from the immunoassay, wherein the different concentrations of the calibration solutions correspond to molar concentrations of the fusion protein without value-assigning the concentrations to determine the concentration of the target protein.

### BRIEF DESCRIPTION OF THE DRAWINGS

FIG. 1 is an example of an assay for NT-proBNP showing value assigned (10×) native synthesized peptide mapping to actual human samples (thus, value assigning these calibrators to 1/10^{th} their actual molar concentration). Also shown in the lower curve the equal molar (not value assigned) native synthesized peptide values.

FIG. 2 is an example of the graphs shown in FIG. 1, with a fusion protein used as a calibrator (e.g., NT-proBNP fused to GST) in the middle curve. This curve may more closely resemble the value-assigned (upper) curve in the presence of detergent, as shown in FIG. 3.

FIG. 3 illustrates the use of detergent to increase the availability of a synthetic peptide, particularly when using a GST fusion.

FIG. 4 schematically illustrates the method of estimating an amount of target protein using a calibrant formed of a synthetic target protein fused to a soluble protein (e.g., GST).

### DETAILED DESCRIPTION

Described herein are method and systems (including compositions of matter and assays including such compositions) for calibration and detection of native proteins in which controls (e.g., recombinant protein controls) are unreliable. In particular, described herein are methods and systems for making a calibrant (control) solution in which a synthetic protein control more closely resembles the native protein. These methods and systems may reduce or eliminate the need for correction (e.g., value assigning) and may therefore enhance the reliability and/or sensitivity of assays using these native and synthetic proteins.

In one variation, the methods and systems described herein use as a control a fusion protein that includes all or a portion (e.g., >80%, greater than 85%, greater than 90%, greater than 95%) of the sequence of the native peptide sequence, fused to a soluble protein, such as Glutathione S-transferase (GST). GST-protein fusions are well-known, however, it is not known or suggested to use such GST-protein fusions as a direct calibrant in an assay for a native protein.

In general, although it may be most desirable to use native protein as a control (calibrant) in immunoassays, the under-recovery of native peptides restricts the effectiveness and/or reduces the desirability of using these peptides as either as a calibrant or control material in assays such as immunoassay or activity assays. Thus, in many instances the use of recombinant form(s) of the native protein may instead be desirable. As mentioned above, however, recombinant proteins may be less available for interaction with immunohistochemical markers (e.g., antibody) than native foul's of proteins, even in the presence of one or more agents (such as detergents) that disaggregate the proteins.

As described herein, the combination of a fusion protein in which all or most of the native protein sequence has been fused with the peptide may be used with a disaggregation agent to provide a more sensitive and/or accurate calibrant and/or control. This improved availability of critical peptide epitopes in the fused peptide either in horse serum or in diluents containing additional disaggregation agents will improve the assay signal to the extent that they could be used as assay calibrators and controls.

In the methods and systems described herein, a peptide (referred to as the target peptide) which is known to aggregate (thereby blocking or masking many native antigenic epitopes) is fused to a soluble protein. This fusion is typically accomplished at the DNA level, and the fusion is subsequently expressed in E. coli, however the same outcome would be accomplished post-translationally, by fusing the purified soluble protein to a modified target peptide using sortase or other protein combinatory (ligation) system.

For example, an N-terminal fragment of the human B-type natriuretic peptide or Ventricular Natriuretic Peptide (NT-proBNP target peptide) may be fused to Glutathione S-transferase (GST) from a parasitic helminth, Schistosoma japonica. Glutathione S-transferase is a 211 amino acid protein (26 kDa) whose DNA sequence is frequently integrated into expression vectors (such as the pGEX expression vector series) for production of recombinant proteins. This recombinant construct will also be engineered with a polyhistidine-tag (6 histidine sequence often referred to as His6 tag (SEQ ID NO: 3)) to aid in the downstream purification of the protein, and with a Tobacco Etch Virus nuclear inclusion a endopeptidase cleavage site (TEV site) allowing the separation of the target peptide from the GST moiety (if desired in subsequent studies). It should be noted that while the His6 Tag (SEQ ID NO: 3) and TEV cleavage sites may be added to this recombinant construct, their presence is not essential or germane to this invention.

This insertion of the target peptide sequence into the expression vector is accomplished in such a way as to result in the fusion of the GST protein to either the C or the N-terminus of the target peptide. Both expression proteins will be evaluated to establish if one orientation is preferable to another within this example. It is expected that the presence of any orientation preference will be dependent upon the sequence of the target peptide.

The expressed protein may be purified using a Ni-NTA column (eluted with 50-300 mM Imidazole) and with a Q-sepharose anion exchange chromatography. The purified GST-NT-proBNP fused protein may be referred to as the “fused peptide” in this description. The native (unfused) synthetic/recombinant NT-proBNP peptide may be referred to as the “native peptide” or native (synthetic) peptide or synthetic native peptide. Purified peptide may be referred to as native (purified) peptide or purified native peptide.

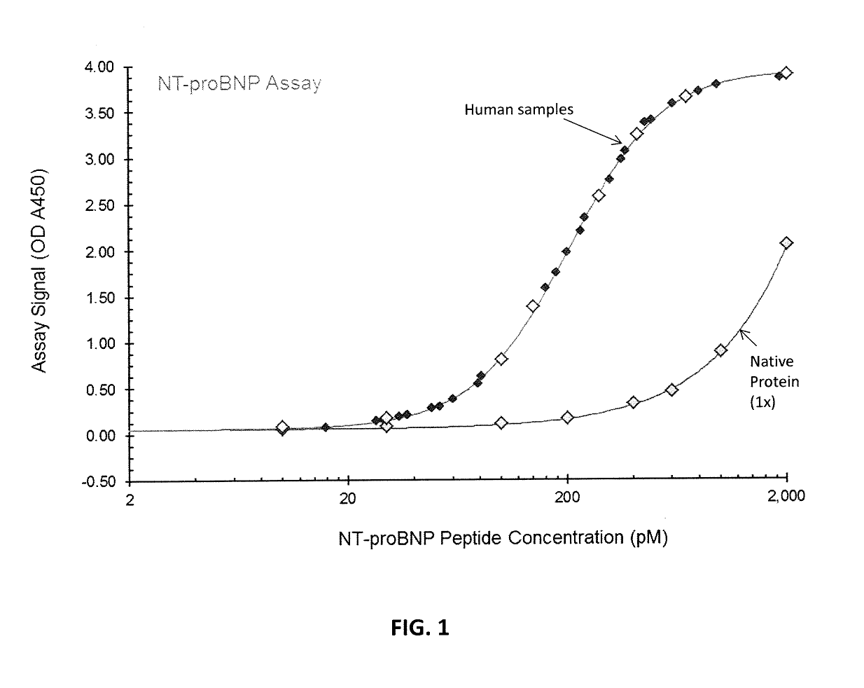

When the native peptide is dissolved in either water or PBS based buffer and subsequently diluted in a horse serum diluent, equal molar amounts of peptide typically will not respond to the expected activity levels in a NT-proBNP EIA assay. In this we mean, as an example, that when 20 pM of native peptide (synthetic native peptide) is added to this assay as an independent sample, the signal is less than the signal of a native (purified) samples containing 20 pM of pro-BNP. In this scenario, the native (synthetic) peptide under-recovery is will be observed across the dynamic range of the NT-proBNP EIA assay. FIG. 1 demonstrates what we expect to observe with a NT-proBNP peptide and assay system.

In FIG. 1, the NT-proBNP Assay is predicted to show an assay signal for an equal molar amount of native (synthetic) peptide; thus, to use the synthetic peptide as a calibrant or control, the concentration of synthetic native peptide must be manipulated. For example, these peptide samples can be augmented by adding more peptide, but retaining the original peptide concentration values. In FIG. 1, the lower trace shows the detected signal based on the synthetic native peptide. Adding 10 times more peptide to each sample (light points, upper trace) results and scaling, or in effect causing these samples to be “Value Assigned” a peptide concentration 1/10 of the actual amount of peptide present, may correct for this discrepancy. When these augmented samples are introduced into the assay, the samples will resemble more closely native (purified) samples. This observation demonstrates that although the peptide contains the antigenic epitopes required to be detected by the EIA assay employed, only a small proportion of these epitopes (sub-equal molar levels) are available to participate in the immuno-reaction(s).

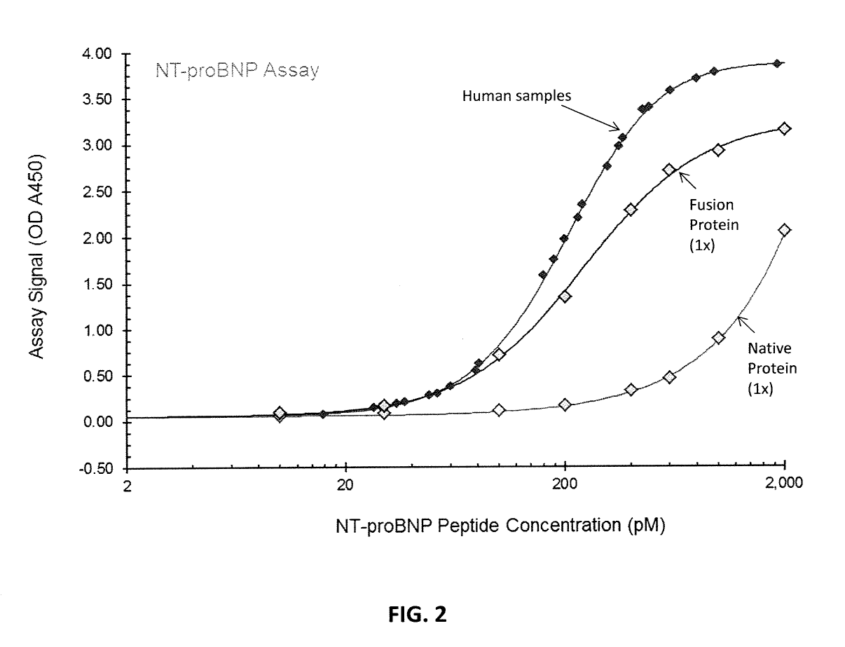

In contrast, fused peptides that is fused to a non-aggregating (e.g., solubilizing) sequence such as GST, as described herein may result in a profile that is closer to that seen with native (purified) peptide. For example, when the fused peptide is substituted for the native peptide as described above, more signal may be obtained at each of the peptide concentrations evaluated. See, e.g., the middle trace in FIG. 2 (the red line). In this example the same molar quantities of either the native or fused peptide are shown as samples in the EIA assay.

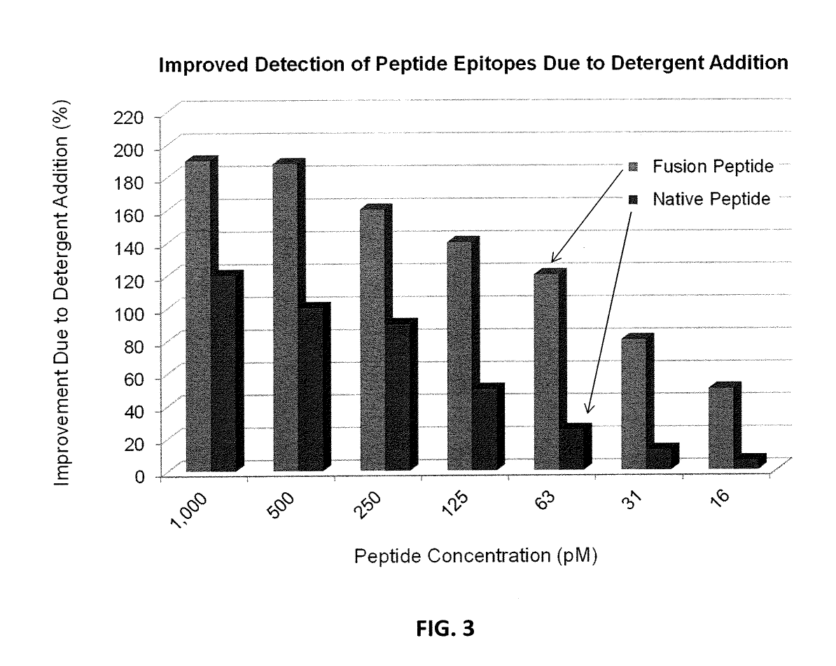

This effect may be even more closely enhanced by using as a calibrant (and/or control) a biochemical agents which are known to aid in the disaggregation of proteins, such as a detergent, when using the fusion peptide. These may include anionic surfactants (such as Sodium dodecyl sulfate or SDS), nonionic surfactants (such as Triton X-100, NP-40 or IGEPAL CA-630), zwitterionic detergents (such as CHAPS) and chaotropic agents (such as Guanidine Hydrochloride). These agents may have less impact upon aggregation state (and subsequent availability of epitopes) with native peptides and even with synthetic native peptide than that observed upon the aggregation state of the fused (synthetic) peptide. These agents may be far more effective at reducing the aggregation state of the fusion peptide). For example, FIG. 3 illustrates an expected differential impact upon peptide based antigenic epitopes with a nonionic surfactant.

Thus, in FIG. 3, at the effects of the use of a detergent on detection (e.g., immunohistochemical detection) of fusion protein vs. native (synthetic) peptide, e.g., relative to native (purified) peptide, is shown for different peptide concentrations. Although the examples provided above are specific to NT-proBNP and GST, additional or alternative embodiments of this invention may include (but are not limited to): NT-proBNP and/or BNP natriuretic peptides, Amyloid peptide (AB42) correlated with Alzheimer's Disease and MR-proANP and/or ANP natriuretic peptides.

In practice, the methods described herein may include a method of performing an immunoassay to detect a native protein (e.g., NT-proBNP, MR-proANP, etc.) wherein the control and/or calibration solutions are prepared using a synthetic version of the native protein to which a solubilizing sequence (e.g., GST) has been added, and preparing the control/calibration solution in the presence of (or in some variations in the presence of a high concentration of) detergent.

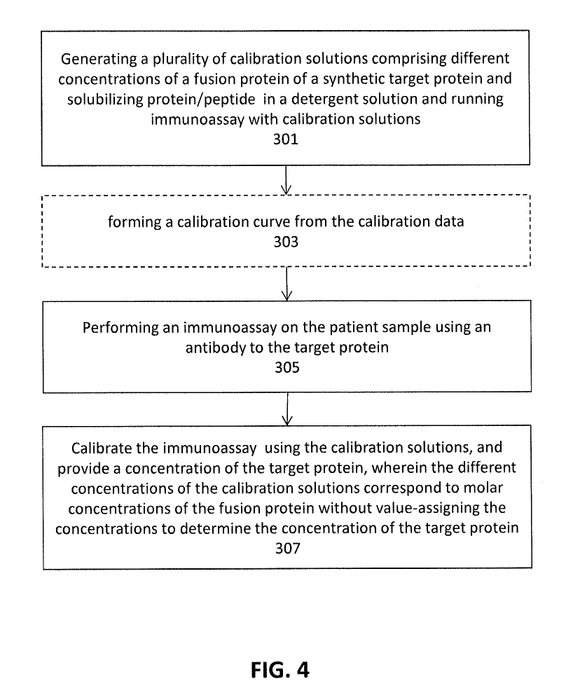

FIG. 4 generically illustrates a method of quantifying a target protein from a patient sample using a synthetic calibration protein having increased solubility. In FIG. 4, the method may include performing an immunoassay on the patient sample using an antibody to the target protein. For example, a blood sample may be used as the patient sample and used in an EIA assay 305. Before, during or after running the immunoassay on the blood sample, a plurality of calibration solutions comprising different concentrations of a fusion protein of a synthetic target protein and a solubilizing protein/peptide (e.g., Glutathione S-transferase (GST)) in a detergent solution may be prepared 301. The immunoassay may be calibrated by performing the immunoassay on these calibration solutions. Optionally a calibration curve may be calculated 303, or a processor may directly use the calibration data. Thus, the plurality of calibration solutions may be used to calibrate the immunoassay and provide a concentration of the target protein, wherein the different concentrations of the calibration solutions correspond to molar concentrations of the fusion protein without value-assigning the concentrations to determine the concentration of the target protein 307.

### NT-proBNP Assay Example

For example, an assay may be configured and performed as described above. N-terminal pro-brain (or B-type) natriuretic peptide (NT-proBNP) is produced predominately by the cardiac ventricular myocytes, and is released in response to volume expansion and filling pressure and is involved in maintaining intravascular volume homeostasis. After synthesis, the peptide is cleaved first to proBNP and subsequently to BNP (active form) and NT-proBNP (inactive form). Natriuretic peptide (NP) levels (BNP and NT-proBNP) are widely used in clinical practice and cardiovascular research as a diagnotic tool for the occurrence and severity of heart failure (HF) and coronary syndrome.

Elevated plasma levels of BNP and NT-proBNP have been observed at times of cardiac stress and damage. It has also been shown that increased NP values in patients with renal dysfunction can suggest the presence of cardiac disease. Low circulating NP levels have been observed in obese people, however the prognostic capacity of these biomarkers were not affected for those patients. Thus, NP levels are quantitative plasma biomarkers of an accurate diagnosis of heart failure. Measurements of NP levels may help in risk stratification of patients suffering heart attacks in emergency care and in accurate and rapid diagnosis of heart failure in primary care.

A sandwich enzyme immunoassay may be performed using an antibody to N-Terminal ProBNP. In a colorimetric assay, a microtiter plate may be pre-coated with an antibody specific to N-Terminal Pro-Brain Natriuretic Peptide (NT-ProBNP). Standards or samples may then added to the appropriate microtiter plate wells with a biotin-conjugated antibody specific to N-Terminal Pro-Brain Natriuretic Peptide (NT-ProBNP). In some variations, Avidin conjugated to Horseradish Peroxidase (HRP) may be added to each microplate well and incubated. After TMB substrate solution is added, only those wells that contain N-Terminal Pro-Brain Natriuretic Peptide (NT-ProBNP), biotin-conjugated antibody and enzyme-conjugated Avidin will exhibit a change in color. The enzyme-substrate reaction may be terminated by the addition of sulphuric acid solution and the color change is measured spectrophotometrically at, e.g., a wavelength of 450 nm±10 nm. The concentration of N-Terminal Pro-Brain Natriuretic Peptide (NT-ProBNP) in the samples is then determined by comparing the O.D. of the samples to the standard curve.

The composition of the standards (and/or calibrators) may be performed using a fused protein, as described above.

For example, the sequence of the NT-proBNP: MDPQTAPSRA LLLLLFLHLA FLGGRSHPLG SPGSASDLET SGLQEQRNHL QGKLSELQVE QTSLEPLQES PRPTGVWKSR EVATEGIRGH RKMVLYTLRA PRSPKMVQGS GCFGRKMDRI SSSSGLGCKV LRRH (SEQ ID NO: 1)

This sequence may be fused (at either end, and/or with a spacer sequence, including His-tags, and cleavage sites) with a GST protein sequence (e.g., GST tagged). An exemplary GST tag may include: MSPILGYWKI KGLVQPTRLL LEYLEEKYEE HLYERDEGDK WRNKKFELGL EFPNLPYYID GDVKLTQSMA IIRYIADKHN MLGGCPKERA EISMLEGAVL DIRYGVSRIA YSKDFETLKV DFLSKLPEML KMFEDRLCHK TYLNGDHVTH PDFMLYDALD VVLYMDPMCL DAFPKLVCFK KRIEAIPQID KYLKSSKYIA WPLQGWQATF GGGDHPPKSD LV (SEQ ID NO: 2)

Using a fusion protein as described above, along with an agent to prevent or limit aggregation (including a detergent), calibration solutions of equimolar or near-equimolar amounts of recombinant protein may be used.

Any of the methods described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. Thus, the calibration methods describe herein may be at least partially implanted with the use of a processor.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Sequence Listings:
NT-proBNP, SEQ ID NO: 1:
MDPQTAPSRA LLLLLFLHLA FLGGRSHPLG SPGSASDLET
SGLQEQRNHL QGKLSELQVE QTSLEPLQES PRPTGVWKSR
EVATEGIRGH RKMVLYTLRA PRSPKMVQGS GCFGRKMDRI
SSSSGLGCKV LRRH
GST tag, SEQ ID NO: 2:
MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRLCHK TYLNGDHVTH PDFMLYDALD
VVLYMDPMCL DAFPKLVCFK KRIEAIPQID KYLKSSKYIA
WPLQGWQATF GGGDHPPKSD LV
His6 Tag, SEQ ID NO: 3:
HHHHHH

What is claimed is:

1. A method of quantifying a target protein from a patient sample using a synthetic calibration protein having increased solubility, the method comprising:
performing an immunoassay on the patient sample using an antibody to the target protein;
generating a plurality of calibration solutions comprising different concentrations of a fusion protein of a synthetic target protein and Glutathione S-transferase (GST) in a detergent solution; and
using the plurality of calibration solutions to calibrate the immunoassay and provide a concentration of the target protein, wherein the different concentrations of the calibration solutions correspond to molar concentrations of the fusion protein without value-assigning the concentrations to determine the concentration of the target protein.

2. The method of claim 1, further comprising using the plurality of calibration solutions to make a calibration curve.

3. The method of claim 1, wherein the immunoassay is a solid-phase enzyme immunoassay (EIA).

4. The method of claim 1, wherein the patient sample is a blood sample.

5. The method of claim 1, wherein the target protein is NT-proBNP.

6. The method of claim 1, wherein the target protein is β Amyloid peptide (AB42).

7. The method of claim 1, wherein the target protein is MR-proANP.

8. The method of claim 1, wherein the fusion protein comprises a His6 Tag (SEQ ID NO: 3).

9. The method of claim 1, wherein the fusion protein comprises a TEV cleavage site between the target protein and the GST.

10. The method of claim 1, wherein the fusion protein comprises the GST fused to the C-terminus of the target protein.

11. The method of claim 1, wherein the fusion protein comprises the GST fused to the N-terminus of the target protein.

12. The method of claim 1, wherein the detergent solution comprises one or more of: an anionic surfactant, a nonionic surfactant, a zwitterionic detergent, and a chaotropic agent.

13. The method of claim 1, wherein the detergent solution comprises one or more of: Sodium dodecyl sulfate (SDS), Triton X-100, Tergitol-type NP-40 (NP-40), octylphenoxypolyethoxyethanol (IGEPAL CA-630), 3-[(3-Cholamidopropyl)dimethylammonio]-1-propanesulfonate (CHAPS), and Guanidine Hydrochloride.

14. A method of quantifying a target protein from a patient sample using a synthetic calibration NT-proBNP having increased solubility, the method comprising:
performing an immunoassay on the patient sample using an antibody to NT-proBNP;
generating a plurality of calibration solutions comprising different concentrations of a fusion protein of a synthetic NT-proBNP and Glutathione S-transferase (GST) in a detergent solution; and
using the plurality of calibration solutions to calibrate the immunoassay and provide a concentration of the NT-proBNP from the immunoassay, wherein the different concentrations of the calibration solutions correspond to molar concentrations of the fusion protein without value-assigning the concentrations to determine the concentration of the target protein.


SEQUENCE LISTING (3 sequences)

SEQ ID NO: 1 - protein, 134 aa, Artificial Sequence
  Description of Artificial Sequence Synthetic polypeptide
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 222 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLV

SEQ ID NO: 3 - protein, 6 aa, Artificial Sequence
  Description of Artificial Sequence Synthetic 6xHis tag
HHHHHH